(19) United States
(12) Patent Application Publication
(10) Pub. No.: US 2012/0301956 A1
(43) Pub. Date: Nov. 29, 2012

(54) Novel hydroxylated enantiomers of (-) 3a,6,6,9a-tetramethylperhydronaphtho[2,1-b]furan as perfuming agents derived from a fungal fermentation process

(76) Inventors: Attaur Rahman, Karachi (PK); Mohammad Iqbal Choudhary, Karachi (PK); Syed Ghulam Musharraf, Karachi (PK); Amtul Sami, Karachi (PK)

(21) Appl. No.: 13/115,991

(22) Filed: May 26, 2011

(51) Int. Cl. C12N 5/07; C12N 5/04; C12N 1/00; B01D 36/02

(52) U.S. Cl. 435/325; 210/150; 210/749; 435/410; 435/243

(57) ABSTRACT

(−)3a,6,6,9a-tetramethylperhydronaphtho[2,1-b]furan (ambrox) is a strong aromatic compound used widely in a variety of perfumery applications and is highly prized for its musky odor. We report novel polar metabolites of (−)3a,6,6,9a-tetramethylperhydronaphtho[2,1-b]furan prepared by a novel process of microbial fermentation using a fungi, Fusarium lini, of unique structures that would be difficult to predict opening up the possibilities of their chemical synthesis. The polar metabolites discovered have stronger aromatic characteristics and offer a new highly prized odiferous characteristic quite different from that of the parent compound and thus can be used in the preparation of perfumes, odor-masking and other odor-management applications.

### FIELD OF INVENTION

The present invention relates to the field of perfumery. It provides in particular a new process for the preparation of polar metabolites of compound I that have stronger and unique olfactive characters of ambergris type with distinct power of diffusiveness.

### BACKGROUND

The legendary amber (Fr. ambergris, grey amber) is a pathological metabolite of the sperm whale, Physeter catodon (Physeteridae), probably arising from injuries in its intestines as a result of certain food intakes. It is abundant in steroid lipids, the tricyclic triterpene (−)-ambrein being one of the main constituents. When the excreted chunks of amber, some weighing as much as 100 kg, are exposed to sunlight and air at the surface of the sea, a number of oxidation products are gradually formed. These compounds have a pronounced odor, highly valued in perfumery since antiquity. The most important amber odorant is (−)-ambrox. Today, it is synthesized from the diterpene sclareol, found in the plant Salvia sclarea (Labiatae), commonly known as Clary sage.

The powerful and elegant odor of (−)-ambrox is somewhat reminiscent of that of chopped bark from pine. According to Müller and Lamparsky (Müller P M, Lamparsky D. Perfumes: Art, Science & Technology. Amsterdam, N.Y.: Elsevier; 1991) it matches the first four tonalities of aged ambergris tincture: wet mossy forest soil, strong tobacco, balsamic sandalwood and warm animal musk (seaweed/ocean and fecal). (−)-Ambrox of high quality is marketed as Cetalox® by Firmenich (Switzerland). An example of a perfume using (−)-ambrox is Drakkar Noir (marketed by Guy Laroche company in 1982). Several odorants have been developed sharing the ambery odor characteristics such as those derived from cedrene, cedrol, (+)-3-carene, or from the family of polycyclic aromatic musks. However, although effective in their own way, none of them compete with (−)-ambrox in aroma and appeal.

### SUMMARY OF INVENTION

This invention describes novel perfumes and a new process of manufacturing the same using fungal fermentation of a steroidal natural products (−)-ambrox (I) leading to the identification, isolation and characterization of three novel metabolites, which were surprisingly more aromatic than ambrox (I) and thus constitute a useful discovery in the field of perfumery. More specifically, this invention claims mono-, di and tri-hydroxylated (−)-ambrox (I): 1α-hydroxyambrox (II), 1α,11α-dihydroxyambrox (III), 1α,6α-dihydroxyambrox (IV), and 1α,6α,11α-trihydroxyambrox (V). These novel compounds are produced by a surprising discovery of enantiomeric hydroxylation reaction that is useful in producing aromatic structures.

### BRIEF DESCRIPTION OF DRAWINGS

FIG. 1: Chemical structure of Compound I showing various substituents.

FIG. 2: Chemical structures of Compounds II-V showing various substituents.

FIG. 3: Scheme of conversion of Compound I upon fermentation

FIG. 4: NMR Interaction of Compound II

FIG. 5: NMR Interaction of Compound III

FIG. 6: NMR Interaction of Compound IV

FIG. 7: NMR Interaction of Compound V

FIG. 8: Selected NOESY Correlations of Compound V

### DETAILED DESCRIPTION OF THE INVENTION

Ambergris has been greatly valued from ancient times. It is now used as a fixative in perfumes and is one of the most valuable animal perfumes, ranked with Civet and Musk [Tamura, H. and Hasegawa, Karyo Gizyutsu Report (Japanese), 1996, 3, 14; Tanimoto, H. And Oritani, T. Tetrahedron 1977, 53, 3527]. Its active principle is ambrein, a crystalline triterpene alcohol with the empirical formula C₃₀H₅₁OH that possesses an amber-like odor [Stoll, M. and Hinder, M. Helv. Chim. Acta, 1950, 33, 1251]. During drifting in the sea for many years, ambrein is oxidatively decomposed by the action of sea water, air and sunlight to give rise to several odorous compounds [Tanimoto, H., Oritani, T. Tetrahedron 1977, 53, 3527; Mori, K., Tamura, H. Leibigs Ann. Chem. 1990, 361-368]. Among these compounds, (−)3a,6,6,9a-tetramethylperhydronaphtho[2,1-b]furan (I) has a strong amber-like odor.

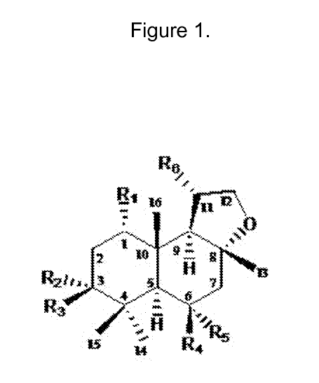

FIG. 1: Chemical Structure of Compound I (C₁₆H₂₈O): R¹=R²=R³=R⁴=R⁵=R⁶=H

It has been reported that the release of ambergris scent is strongly related to triaxial conformation of C-8 and C-10 methyl groups, which play an important role in the hydrophobic interactions with the hypothetical receptor site. (−)-3a,6,6,9a-tetramethylperhydronaphtho[2,1-b]furan (I) is found to be a much stronger perfume than the (+)3a,6,6,9a-tetramethylperhydronaphtho[2,1-b]furan. In recent years, since it became more difficult to obtain many kinds of animal perfumes because of the gradual reduction in the world's wild resources and the conservation of wild animals, considerable attention has been paid to the total chemical synthesis of compound I.

A completely synthetic form of Compound I is prepared as follows: (−)-2,5,5,8a-Tetramethyl-1-(carboxymethyl)-2-hydroxydecalin is subjected to lactonization by dehydration to form decahydro-3a,6,6,9a-tetramethyl(3aα,5aβ,9aα,9bβ)-(+)-naphtho[2,1-b]furan-2(1H)-one, which is then reduced with a metal hydride to convert it into (−)-2,5,5,8a-tetramethyl-1-(carboxymethyl)-2-hydroxydecalin, followed by dehydrative cyclization to yield a racemic mixture which is then resolved using a 1-(aryl)ethylamine to give pure I. (U.S. Pat. No. 5,290,955 issued to Asanuma, et al., Mar. 1, 1994.)

In the field of the synthesis of compounds having musky odors, there has been great activity for the last ten years, resulting from the need to find novel musky odor compounds which can replace certain compounds of widespread use in perfumery and which use is becoming more and more restricted due to toxicological and ecological reasons. The esters according to the present invention are products which fulfill the requirements for perfuming compounds, and they are capable of replacing the above-mentioned known compounds.

Because of the highly prized odor of compound I, considerable work has been done in the recent past to develop compounds of similar structure to provide more potent or different perfumes; chemical derivatization and microbial fermentation are two techniques that have been used to prepare compounds that will have similar musk odor but of different notes. Using the technique of fermentation, several metabolites of Compound I have been earlier reported upon fermentation with Cephalosporium aphidicola (wild type), Aspergillus niger (IFO 4049) and Aspergillus cellulose (IFO 4040) [Hanson, J. R. and Truneh, A. Phytochemistry 1998, 142(4), 1021; Hashimoto, T., Noma, Y., Asakawa, Y. Heterocycles 2001, 54(1), 529; Farooq, A., Tahara, S. Z. Naturforsch. 2000, 55, 341-346.

The polar metabolites of compound I claimed in this invention are stronger and more powerful in eliciting an olfactory response. What was most surprising in our invention was the observation that the presence of a hydroxyl group gives rise to unique odoriferous properties of compound I which are clearly distinct from those of the parent non-hydroxylated compound. Their odor can be described as a woody note, together with an intense fruity note. The quality and intensity of said fruity note can be of varied nature, but the woody bottom note is always clearly present, rendering said compounds particularly useful in perfumery.

The present invention relates more particularly, to hydroxyl enantiomers derived from compound I by subjecting it to a fermentation process using a fungus, Fusarium lini that has never been used in any prior art to cause fermentation of compound I. We have found that the hydroxyl enantiomers identified and characterized in the present invention (compounds II-V).

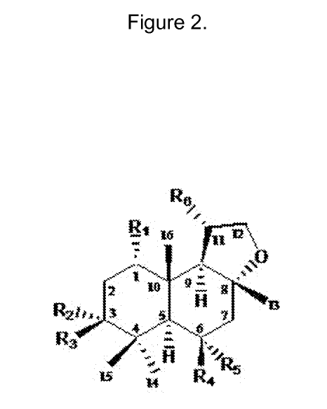

FIG. 2: Chemical Structure of Compounds II-V

Compound II: [9α-Hydroxy-dodecahydro-3,6,6,9a-tetramethyl-naphtho-[2,1-b]furan]: R²=R³=R⁴=R⁵=R⁶=H; R¹=OH

Compound III: [(9α,1α-Dihydroxy-dodecahydro-3,6,6,9a-tetramethyl-naphtho-[2,1-b]furan]: R²=R³=R⁴=R⁵=H; R¹=R⁶=OH

Compound IV: [9α,5α-Dihydroxy-dodecahydro-3,6,6,9a-tetramethyl-naphtho-[2,1-b]furan]: R¹=R²=R³=R⁴=H; R¹=R⁵=OH

Compound V: [9α,5α,1α-Trihydroxy-dodecahydro-3,6,6,9a-tetramethyl-naphtho-[2,1-b]furan]: R²=R³=R⁴=H; R¹=R⁵=R⁶=OH

Modern spectroscopic techniques, including two-dimensional NMR, single X-ray diffraction and mass spectrometry, were employed for the structure elucidation of new metabolites. In this invention, we have carried out microbial transformation of compound I to obtain many new mono-, di- and tri-hydroxylated metabolites, which were otherwise difficult to synthesize by using conventional chemical methods and therefore impossible to predict. The most surprising discovery reported here is the enantioselective a-hydroxylation that occurred at C-1, C-6 and C-11; this reaction has never been reported in the scientific literature. We further find that compounds II-V have musky odors which are quite distinct from those of the parent compound I.

Of course, in spite of the fact that the compounds of the present invention possess a common odor of the woody-fruity type, there are differences between the various products (among compounds II-V), and these can be quite pronounced. As a result, compounds II-V can be widely used in fine perfumery either as pure isomers, alone or in a mixture of any the compounds II-V, combined with compound I or other odiferous compounds.

The field of this invention is perfumery. The object of the present invention is to provide new compounds which are useful as perfume ingredients, to impart odors of the musky type. This object is attained by the discovery of new compounds, a new method of manufacturing them and suggested compositions of commercial value.

The object is achieved wherein compounds II-V which are polar metabolites of formula I are identified, isolated and purified upon fermentation in a fungus. These hydroxyl enantiomers esters (compound II-V) are novel compounds. The compounds of the invention can be used in practically all fields of modern perfumery. There can be cited here applications in fine perfumery, namely for the preparation of perfumes and colognes in which original olfactive effects can be obtained.

Another object of the invention is to describe a method or process of manufacturing hydroxylated enantiomers of I using fermentation in a fungal culture.

Another object of the invention is the use of the compounds of the above formulae in perfumery, as well as the perfumes perfuming compositions and perfumed articles containing these compounds.

The compounds can also be used in functional perfumery. Non-limiting examples for this type application include soaps, bath and shower gels, shampoos and other hair care products, deodorants and an antiperspirants, air fresheners, liquid and solid detergents for the treatment of textiles, fabric softeners, or all purpose cleaners. In these applications, the compounds (II-V) can be used alone or in admixture with other perfuming ingredients, solvents or adjuvants of current use in perfumery.

The nature and the variety of other ingredients do not require a more detailed description here, which, moreover, would not be exhaustive, and the person skilled in the art will be able to choose the latter through its general knowledge and as a function of the nature of the product to be perfumed and of the desired olfactive effect. These perfuming ingredients belong to chemical classes as varied as alcohols, aldehydes, ketones, esters, ethers, acetates, nitrites, terpene hydrocarbons, sulfur- and nitrogen-containing heterocyclic compounds, as well as essential oils of natural or synthetic origin. A large number of these ingredients are listed in reference textbooks such as the book of S. Arctander, Perfume and Flavor Chemicals, 1969, Montclair, N.J., USA, or its more recent versions, or in other works of similar nature.

The compounds of the invention can be present in the form of the four enantiomers in the pure state or as a mixture of the enantiomers. The invention thus includes all these possible mixtures and all the possible individual isomers. As the olfactive note of each of these isomers can of course be different from that of the others, the odor of every possible isomer mixture can also change as a function of the content of any given enantiomer.

Another object of the invention is to describe a method of selective hydroxylation of compound I using fermentation in a fungal culture.

### Method of Manufacture

The compounds of the invention are generally prepared by fermentation of compound I by a fungus, however, other methods involving other microorganisms, chemical methods may also be used and are thus included in the process claimed in this invention.

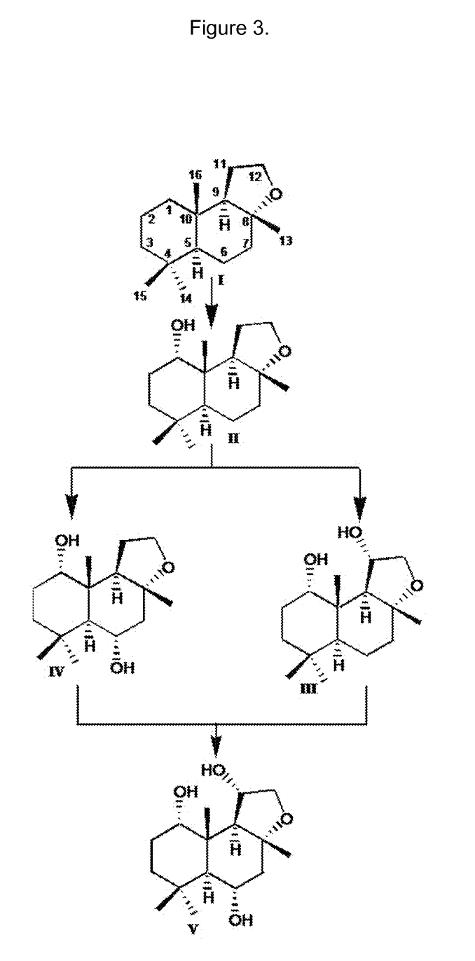

Fermentation process is conducted using Fusarium lini. The medium for Fusarium lini (ATCC 9593) includes the following chemicals dissolved in distilled H₂O (3 L): glucose (30.0 g), glycerol (30.0 g), peptone (15.0 g), yeast extract (15.0 g), KH₂PO₄ (15.0 g), and NaCl (15.0 g). Stage I liquid cultures were prepared by inoculating the spores from the well-grown Fusarium lini (ATCC 9593) on the agar slants into the conical flasks (250 mL), containing 100 mL of sterilized medium. The flasks were then incubated on a shaker table for two days (48 hr). Stage II cultures were prepared by transferring 1 ml of the stage I mycelia suspension into conical flasks containing medium (100 mL each). Stage II Fermentation Protocol [Smith, R. V. and Rosazza, J. P. J. Pharm. Sci. 1975, 64, 1737] was used for all biotransformational studies. Compound I (600 mg), dissolved in 15 mL of acetone, was evenly distributed among 30 flasks containing stage II culture; fermentation stopped after 8 days. After filtration, extraction and evaporation the medium afforded a brown gum (1.63 gm) and after repeated column chromatography over silica gel, with gradient fractions of petroleum ether and ethyl acetate, afforded metabolites II-V (see scheme below, FIG. 3).

FIG. 3: Scheme of Conversion of Compound I Upon Fermentation

### Purification and Characterization of Compounds II-V:

Compound II: 9α-Hydroxy-dodecahydro-3,6,6,9a-tetramethyl-naphtho-[2,7-b]furan elution was carried out with 28% ethyl acetate—62% petroleum ether, which provided II (16 mg, 2.6%) was crystallized from 100% methanol (MeOH) to give white needles.

M.P. 101-102° C.

U.V. (MeOH) λₘₐₓ nm (log ε): 201 (2.5), λₘᵢₙ nm (log ε): 192 (1.6).

[α]²⁵_{D}: 1.72 (c=0.1, MeOH).

IR (CHCl₃) νₘₐₓ: 3436 (OH), 2938 (CH), 1380 (C-O) cm⁻¹.

¹H-NMR (CDCl₃, 500 MHz): See Table 1

¹³C-NMR (CDCl₃, 125 MHz): See Table 1

EI MS m/z (% rel. int.): 252 [M]⁺ (17), 237 [M−Me]⁺ (100%), 219 [M-Me-H₂O]⁺ (54), 163 (4), 152 (14), 111 (19), 97 (37), 55 (69).

HREI MS m/z (formula, calculated): 252.2056 (C₁₆H₂₈O₂, 252.2089).

### NOE Experiments:

Irradiation at δ 0.82 (Me-16β/15β): n.O.e. at H-1 (δ 3.46) (9.2%).

Irradiation at δ 1.07 (Me-13β): n.O.e. at H-1 (δ 3.46) (10.5%).

Compound II was obtained as a colorless crystalline solid and characterized as a 1α-hydroxyambrox (II) through spectroscopic studies. The EI MS showed the molecular ion at m/z 252, 16 a.m.u. higher than compound I; while the loss of a water molecule yielded an ion at m/z 234, indicating the presence of a hydroxyl group. The HREI MS of II displayed the molecular ion at m/z 252.2056, corresponding to the formula C₁₆H₂₈O₂ (calculated 252.2089), with one additional oxygen atom. The UV spectrum of II showed only terminal absorption at 201 nm, indicating the lack of chromophore in the molecule. Absorption at 3436 cm⁻¹ in the IR spectrum of II indicated the presence of a hydroxyl group. The ¹H-NMR spectrum of II, indicated the formation of monohydroxy derivative of I, by the appearance of a geminal proton at δ 3.46 (t, J_{1eq,2eq,ax}=2.6 Hz). The splitting pattern and coupling constant of this methine signal indicated that the OH group must be axially oriented and could be situated either at C-1, C-3 or C-7.

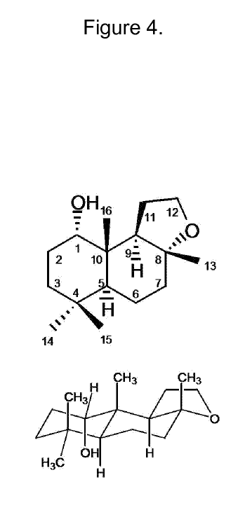

FIG. 4: NMR Interactions of Compound II

The ¹³C-NMR of compound II was very informative; indicating the presence of an additional methine carbon which resonated at δ 72.4, along with the γ-upfield shifts of a number of carbon signals including C-3, C-5 and C-9 as compared to I. This indicated the location of a new hydroxyl group at C-1. The 2D-NMR spectra (HMQC, HMBC and COSY 45°) of compound II were recorded to unambiguously assign the chemical shift values to all the protons and carbons. The ¹³C/¹H connectivities were determined from HMQC spectrum and further confirmed through COSY 45° and HMBC interactions (Table-1.2.2). H-1 (δ 3.46) showed homonuclear couplings with 2-Hₐ (δ 2.1) and 2-H_{b} (δ 1.52) in the COSY 45° spectrum. The HMBC spectrum showed the ³J-heteronuclear interactions of C-1 proton (δ 3.46) with C-3 (δ 35.3) and C-5 (δ 48.8) and thus further supported the position of a new hydroxyl group at C-1. The stereochemistry of the newly introduced hydroxyl group was further investigated by NOED experiments, which showed 10.5% and 9.2% enhancements of signal corresponding to H-1 (δ 3.46), when Me-13β (δ 1.07) and Me-15β/16β (δ 0.82) were irradiated, respectively. This indicated an α disposition (axial) of the hydroxyl group at C-1.

TABLE 1
¹H (500 MHz) and ¹³C-NMR (125 MHz) Chemical Shift
Assignments of 1α-Hydroxyambrox (II) in CDCl₃.

| Carbon | δ_{C} | Multiplicity | δ_{H} (J = Hz) |
|---|---|---|---|
| 1 | 72.4 | CH | 3.46 t (2.6) |
| 2 | 25.6 | CH₂ | 2.10 m; 1.52 m |
| 3 | 35.3 | CH₂ | 1.58 m; 1.21 dd (2.6, |
| | | | 4.1) |
| 4 | 33.0 | C | — |
| 5 | 48.8 | CH | 1.42 m |
| 6 | 20.4 | CH₂ | 1.32 m; 1.17 m |
| 7 | 39.5 | CH₂ | 1.97 m; 1.38 m |
| 8 | 80.3 | C | — |
| 9 | 52.5 | CH | 1.92 dd (2.6, 13.3) |
| 10 | 40.1 | C | — |
| 11 | 22.3 | CH₂ | 1.46 m; 1.17 m |
| 12 | 64.9 | CH₂ | 3.88, 2H m |
| 13 | 21.3 | CH₃ | 1.07 s |
| 14 | 33.3 | CH₃ | 0.90 s |
| 15 | 20.9 | CH₃ | 0.82 s |
| 16 | 15.9 | CH₃ | 0.82 s |

Compound III: 9α,7α-Dihydroxy-dodecahydro-3,6,6,9a-tetramethyl-naphtho-[2,1-b]furan was eluted with 39% ethyl acetate-61% petroleum ether afforded III (8 mg, 1.3%) as a colorless crystalline compound.

M.P. 174-102° C.

[α]²⁵_{D}: −1.53 (c=0.1, MeOH).

U.V. (MeOH) λₘₐₓ nm (log ε): 202 (2.5), λₘᵢₙ nm (log ε): 194 (1.6).

IR (CHCl₃) νₘₐₓ: 3354 (OH), 2941 (CH), 1316 (CO) cm⁻¹.

¹H-NMR (CDCl₃, 500 MHz): See Table 2

¹³C-NMR (CDCl₃, 125 MHz): See Table 2

EI MS m/z (% rel. int.): 268 [M]⁺ (7), 253 [M-Me]⁺ (60), 235 [M-Me-H₂O]±(87), 163 (4.6), 205 (24), 164 (12), 136 (79), 121 (52), 95 (55), 81 (100), 55 (95).

HREI MS m/z (formula, calculated.): 268.2056 (C₁₆H₂₈O₃, 268.2038).

### NOE Experiments:

Irradiation at δ 1.12 (Me-13β): n.O.e. at H-11 (δ 4.51) (7.5%).

Irradiation at δ 4.51 (H-11): n.O.e. at Me-13β (δ 1.12) (3.1%).
n.O.e. at Me-16β (δ 0.82) (3.6%).

Compound III was isolated as a colorless crystalline solid and characterized through detailed spectroscopic study as 1α,11α-dihydroxyambrox (III). The EI MS displayed the molecular ion at m/z 268, 32 a.m.u. greater than that of compound I. The HREI MS showed the molecular ion at m/z 268.2056, consistent with the formula C₁₆H₂₈O₃ (calculated 268.2038), with two more oxygen atoms as compared to I. These preliminary observations indicated dihydroxylation of compound I. The metabolite III showed an IR absorption at 3354 cm⁻¹, indicating the presence of a hydroxyl group. The UV spectrum in methanol displayed only terminal absorptions.

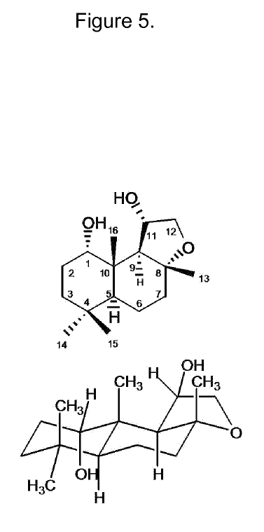

FIG. 5: NMR Interactions of Compound III

The ¹H-NMR spectrum of compound III was found to be remarkably different from I in several aspects. It showed two new methine proton signals resonating at δ 3.69 (t, J_{1eq,2eq,ax}=2.8 Hz) and 4.51 (ddd, J₁₁_{_{β}}_{,9}_{_{α}}=10.2 Hz, J₁₁_{_{β}}_{,12}_{_{α}}=7.1 Hz, J₁₁_{_{β}}_{,12}_{_{β}}=3.9 Hz) while the C-12 methylene protons showed a downfield shifts resonating at δ 4.15 (dd, J₁₂_{_{αβ}}=9.6 Hz, J₁₂_{_{α}}_{,11}_{_{β}}=7.1 Hz) and 3.63 (dd, J₁₂_{_{βα}}=9.6 Hz, J₁₂_{_{β}}_{,11}_{_{β}}=4.1 Hz), indicating the possibility of the presences of a new hydroxyl group at C-11. The ¹³C-NMR spectral data (Table-1.2.3), when compared with compound I, showed two new methine resonances at δ 72.0 and 70.0. The DEPT spectra showed seven CH₂ and four CH, indicating the conversion of two methylene carbons into hydroxy-bearing methines. 2D-NMR spectra (HMQC, HMBC and COSY 45°) of III were recorded to unambiguously assign the chemical shifts to all the protons and carbons. The ¹³C/¹H connectivities were determined from the HMQC spectrum. The signal at δ 3.69 (t, J_{1eq,2eq,ax}=2.8 Hz) could be assigned to C-1β-H. The position of the other hydroxyl group was assigned to be at C-11 on the basis of COSY 45° and HMBC techniques, where H-11 (δ 4.51) showed homonuclear interactions with H₂-12 (δ 4.15 and 3.63) and H-9 (δ 2.11), and heteronuclear interactions with C-12 (δ 73.1).

The stereochemistry of C-11 hydroxyl group was inferred from the NOE-difference experiment, which showed 7.5% enhancement of H-11 signal (δ 4.51) upon irradiation of Me-13β (δ 1.12); similarly irradiation of H-11 signal caused the 3.1% and 3.6% enhancement of signals at δ 1.12 (Me-13β), and 0.82 (Me-16β), respectively, supporting the β-stereochemistry of the C-11 proton. The metabolite III could be formed by the monohydroxylation of II at C-11.

TABLE 2
¹H (500 MHz) and ¹³C-NMR (125 MHz) Chemical Shift
Assignments of 1α,11α-Dihydroxyambrox (III) in CDCl₃.

| Carbon | δ_{C} | Multiplicity | δ_{H} (J = Hz) |
|---|---|---|---|
| 1 | 72.0 | CH | 3.69 d (2.8) |
| 2 | 24.9 | CH₂ | 2.02 m; 1.58 m |
| 3 | 35.5 | CH₂ | 1.26 m; 1.19 m |
| 4 | 33.1 | C | — |
| 5 | 49.0 | CH | 1.5 m |
| 6 | 20.3 | CH₂ | 1.81 m; 1.32 m |
| 7 | 39.9 | CH₂ | 1.84 m; 1.56 m |
| 8 | 82.3 | C | — |
| 9 | 59.0 | CH | 2.1 d (10.2) |
| 10 | 40.3 | C | — |
| 11 | 70.0 | CH₂ | 4.51 ddd (10.2, 7.1, |
| | | | 3.9) |
| 12 | 73.1 | CH₂ | 4.15 dd (9.6, 7.1) |
| | | | 3.63 dd (9.6, 4.1) |
| 13 | 21.9 | CH₃ | 1.12 s |
| 14 | 33.3 | CH₃ | 0.93 s |
| 15 | 20.7 | CH₃ | 0.91 s |
| 16 | 16.7 | CH₃ | 0.82 s |

Compound IV (9α, 5α-Dihydroxy-dodecahydro-3,6,6,9a-tetramethyl-naphtho-[2,1-b]furan) was eluted at 44% ethyl acetate-56% pet ether, as afforded IV (19 mg, 3.1%) white crystalline solid.

M.P. 119-120° C.

[α]²⁵_{D}. −18.7 (c=0.1, MeOH).

U.V. (MeOH) λₘₐₓ nm (log ε): 201 (2.5), λₘᵢₙ nm (log ε): 191 (1.6).

IR (CHCl₃) νₘₐₓ: 3329 (OH), 2931 (CH), 1385 (C-0) cm⁻¹.

¹H-NMR (CDCl₃, 500 MHz): See Table 3

¹³C-NMR (CDCl₃, 125 MHz): See Table 3

EI MS m/z (% rel. int.): 268 [M]⁺ (7), 253 [M-Me]⁺ (100), 235 [M-Me-H₂O]±(66), 163 (4), 217 (9), 191 (24), 167 (7), 139 (13), 111 (21), 81 (24), 55 (62).

HREI MS m/z (formula, calculated): 268.2059 (C₁₆H₂₈O₃, 268.2038).

### NOE Experiments:

Irradiation at δ 4.62 (H-6): n.O.e. at Me-16β (δ 1.02) (4.86%).

Irradiation at δ 1.02 (Me-16β): n.O.e. at H-6 (δ 4.62) (24.5%).
n.O.e. at H-1 (δ 3.42) (2.6%).

Compound IV was obtained as a colorless crystalline solid. The structure of 1α,6α-dihydroxyambrox (IV) was determined on the basis of detailed spectroscopic studies. The EI MS of IV displayed the molecular ion at m/z 268, 32 a.m.u. greater than that of compound I. The HREI MS showed the molecular ion at m/z 268.2059 (C₁₆H₂₈O₃, calculated 268.2038), indicating two additional oxygen atoms than I. The presence of hydroxyl functions was inferred from additional absorption at 3329 cm⁻¹ in the IR spectrum of compound IV. The UV spectrum in methanol displayed a terminal absorption at 201 nm indicating the absence of a chromophore in the molecule.

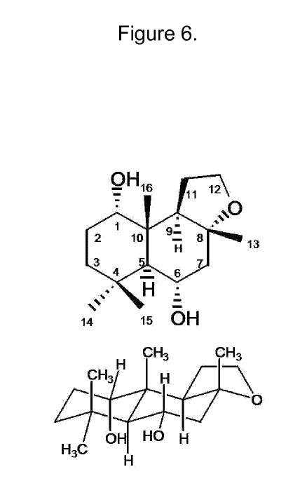

FIG. 6: NMR Interactions of Compound IV

The ¹H-NMR spectrum of IV showed signals for two protons geminal to hydroxyl that resonated at δ 3.42 (t, J_{1eq,2eq,ax}=2.7 Hz) and 4.62 (dd, J_{6ax,5ax}=7.1 Hz, J_{6ax,7eq}=3.0 Hz). The splitting pattern and coupling constants of these signals suggested a orientations of the OH groups at C-1 and C-6 positions. The ¹³C-NMR spectra of compound IV showed disappearance of C-1 and C-7 methylene carbon signals and appearance of two additional hydroxyl-bearing methine signals at δ 74.7 and 71.1 as compared to Compound I (Table-1.2.4). The 2D-NMR spectra (HMQC, HMBC and COSY 45°) of compound IV were recorded to unambiguously assign the chemical shifts to all protons and carbons. The ³C/¹H connectivities were determined from the HMQC spectrum (Table-1.2.4). The 3β-H (δ 3.42) showed interactions with H₂-2 (δ 2.17 and 1.51), while H-6 (δ 4.62) showed COSY° 45 interactions with H₂-6 (δ 2.05 and 1.64) and H-5 (δ 1.58). H-6 also showed heteronuclear interactions (HMBC) with C-8 (δ 80.9) and C-10 (41.5). These observations further supported the position of a new hydroxyl group at C-6. The stereochemistry of C-3 and C-6 methine protons was further investigated by NOE difference measurements between H-6/Me-16β (4.86%), Me-16β/H-6 (24.5%) and H-1/Me-15β (1.16%). The metabolite IV may be formed by the sequential hydroxylation of Compound I into compound IV.

TABLE 3
¹H (500 MHz) and ¹³C-NMR (125 MHz) Chemical Shift
Assignments of 1α,6α-Dihydroxyambrox (IV) in CDCl₃.

| Carbon | δ_{C} | Multiplicity | δ_{H} (J = Hz) |
|---|---|---|---|
| 1 | 74.7 | CH | 3.42 t (2.7) |
| 2 | 25.1 | CH₂ | 2.17 m; 1.51 m |
| 3 | 38.8 | CH₂ | 1.60 m; 1.17 m |
| 4 | 35.3 | C | — |
| 5 | 52.1 | CH | 1.58 d (7.1) |
| 6 | 71.1 | CH | 4.62 dd (7.3, 3.0) |
| 7 | 48.5 | CH₂ | 2.05 m; 1.64 dd |
| | | | (12.1, 3.8) |
| 8 | 80.9 | C | — |
| 9 | 54.0 | CH | 1.87 m |
| 10 | 41.5 | C | — |
| 11 | 27.1 | CH₂ | 1.81 m; 1.47 m |
| 12 | 65.8 | CH₂ | 3.83 dd (16.0, 7.9) |
| | | | 3.92 dd (12.0, 7.8) |
| 13 | 23.9 | CH₃ | 1.31 s |
| 14 | 25.1 | CH₃ | 1.26 s |
| 15 | 23.6 | CH₃ | 1.16 s |
| 16 | 18.4 | CH₃ | 1.02 s |

Compound V: 9α,5α,7α-Trihydroxy-dodecahydro-3,6,6,9a-tetramethyl-naphtho-[2,1-b]furan] was eluted at 71% ethyl acetate-29% petroleum ether afforded V (27.8 mg, 4.6%) as a colorless crystalline compound.

M.P. 147-148° C.

[α]²⁵_{D} −26 (c=0.1, MeOH).

U.V. (MeOH) λₘₐₓ nm (log ε): 203 (2.5), λₘᵢₙ nm (log ε): 195 (1.6).

IR (CHCl₃) νₘₐₓ: 3411 (OH), 2930 (CH), 1317 (C-0) cm⁻¹.

¹H-NMR (CDCl₃, 500 MHz): See Table 4

¹³C-NMR (CDCl₃, 125 MHz): See Table 4

EI MS m/z (% rel. int.): 284 [M]⁺ (2), 269 [M-Me]⁺ (32), 251 [M-(Me+H₂O)]⁺ (93), 218 (11), 215 (13), 175 (9), 139 (23), 109 (99), 81 (69), 55 (100).

HREI MS m/z (formula, calculated): 284.1912 (C₁₆H₂₈O₄, 284.1987).

Metabolite V was isolated as white crystalline solid, and characterized through detailed physical and NMR spectroscopic studies as 1α,6α,11α-trihydroxyambrox (V). The HREI MS of compound V showed the M⁺ at 284.1912 (C₁₆H₂₈O₄, calculated 284.1987). The UV spectrum displayed a terminal absorption at 203 nm, while the IR spectrum displayed absorption at 3411 cm⁻¹, characteristic of hydroxyl group.

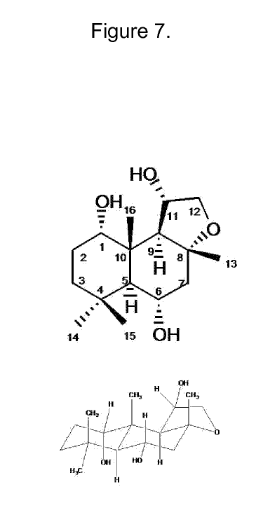

FIG. 7: NMR Interactions of Compound V

The ¹H-NMR spectrum of V exhibited three additional downfield methine proton signals at δ 3.62 (t, J=2.2 Hz), 4.63 (dd, J_{6ax,5ax}=7.4 Hz, J_{6ax,7eq}=2.3 Hz) and 4.59 (ddd, J₁₁_{_{β}}_{,9}_{_{α}}=9.9 Hz, J₁₁_{_{β}}_{,12}_{_{α}}=7.1 Hz, J₁₁_{_{β}}_{,12}_{_{β}}=4.0 Hz), which could be assigned as C-1β, C-6β and C-11β protons, respectively. Analysis of the ¹³C-NMR spectral data showed additional methine carbons resonating at δ 73.2, 69.5 and 71.0, corresponding to OH-bearing C-1, C-7 and C-11, respectively. The position and stereochemistry of the newly introduced hydroxyl groups was further investigated by 2D NMR spectroscopy. COSY 45° spectrum showed couplings between H-1 (δ 3.62)/H₂-2 (δ 2.14, 1.59); H-6 (δ 4.63)/H₂-7 (δ 2.01, 1.77) and H-5 (δ 1.47); H-11 (δ 4.59)/H₂-12 (δ 4.20, 3.61) and H-9 (δ 2.21). This HMBC spectrum showed the heteronuclear interactions between H-1/C-8 (δ 81.3), C-10 (δ 40.6), H-6/C-3 (δ 37.6), C-5 (δ 51.0) and H-1/C-12 (δ 73.0). The splitting pattern of H-1 and H-6 signals indicated their axial (a) and equatorial (a) orientation of the geminal hydroxyl groups, respectively.

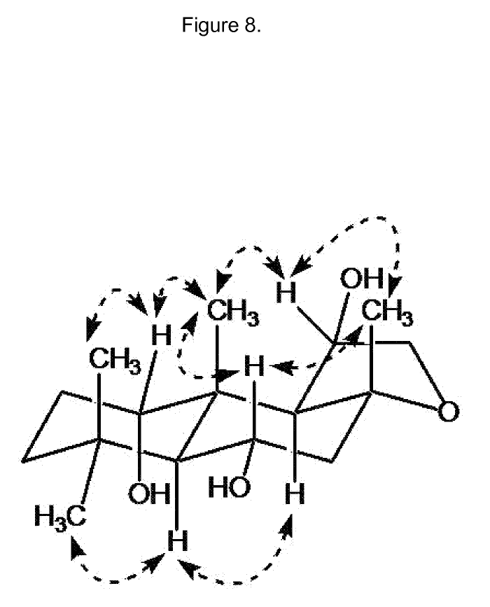

FIG. 8: Selected NOESY Correlations of Compound V

The stereochemistry at C-11 was investigated by NOESY experiments, which showed the β-orientation of C-11 proton. These observations supported the stereochemistry of the metabolite to be 1α,6α,11α-trihydroxyambrox (V).

TABLE 4
¹H (500 MHz) and ¹³C-NMR (125 MHz) Chemical Shift
Assignments of 1α,6α,11α-Trihydroxyambrox (V) in CDCl₃.

| Carbon | δ_{C} | Multiplicity | δ_{H} (J = Hz) |
|---|---|---|---|
| 1 | 73.2 | CH | 3.62 t (2.2) |
| 2 | 25.0 | CH₂ | 2.14, m; 1.59, m |
| 3 | 37.6 | CH₂ | 1.69 m |
| | | | 1.14, ddd (3.6, 6.9, |
| | | | 13.2) |
| 4 | 34.1 | C | — |
| 5 | 51.0 | CH | 1.47, d (7.2) |
| 6 | 69.5 | CH | 4.63 dd (7.4, 2.3) |
| 7 | 48.2 | CH₂ | 2.01 m; 1.77 m |
| 8 | 81.3 | C | — |
| 9 | 59.1 | CH | 2.2 dd (13.2, 2.6) |
| 10 | 40.6 | C | — |
| 11 | 71.0 | CH₂ | 4.59 ddd (4.0, 7.1, 9.9) |
| 12 | 73.0 | CH₂ | 4.20 dd (9.5, 7.1) |
| | | | 3.61 dd (9.4, 4.1) |
| 13 | 23.6 | CH₃ | 1.36 s |
| 14 | 32.8 | CH₃ | 1.25 s |
| 15 | 22.7 | CH₃ | 1.20 s |
| 16 | 17.7 | CH₃ | 1.02 s |

### EXAMPLES

The invention reported here reveals itself appropriate for the preparation of various perfuming compositions, bases and perfuming concentrates, as well as for perfumes and colognes, to which they confer a woody-fruity character of musk type. Their use for the perfuming of various articles, like soaps, bath or shower gels, shampoos, hair-conditioning creams and lotions, cosmetic preparations, body deodorants or air fresheners is also advantageous. Moreover, they are also appropriate for the perfuming of detergents or fabric softeners and of all-purpose household cleaners.

The proportions in which the compounds of the present invention can be used in the various above-mentioned products vary within a wide range of values. These values depend on the nature of the product to be perfumed and the desired olfactive effect. The proportions used also depend on the nature of other ingredients in a given composition, when the compounds of the invention are used in admixture with other perfuming ingredients, solvents or adjuvants of current use in the art.

The compounds of the present invention can of course also be added to the perfuming compositions or perfumed articles either as such or in solution in solvents of current use in the art. As an example, there can be cited concentrations of the order from 1 to 10%, even 20% or more, by weight with respect to the perfuming composition into which they are incorporated. Much lower concentrations than those cited above can be used when the compounds are used for the perfuming of the various products cited above.

Given below are some examples of the compositions as preferred embodiments; however, there are numerous possibilities of formulating perfumes for different purposes as well established in the art and science of perfumery design. The odiferous component described below can consist of either a single compound (II-V) or a single compound in combination of compound I, or any given permutation and combination of compounds II-V with or without compound I. This selection if identified below simply as PERFUME.

### Example 1

### Liquid

| | Parts by |
|---|---|
| Ingredient | weight |
| PERFUME | 60 |
| Linalyl acetate | 350 |
| Lemon oil | 600 |
| Coumarin | 70 |
| 2,6-dimethyl-7-octen-2-ol | 660 |
| Estragon oil | 20 |
| 10%, 2,6,10-trimethyl-9-undecanal | 35 |
| 2-Methyl-4-(2,2,3-trimethyl-3-cyclopenten-1-yl)-4-penten- | 5 |
| 1-ol.sup.6) | |
| 1,3,4,6,7,8-hexahydro-4,6,6,7,8,8-hexamethyl-cyclopenta[g]- | 200 |
| 2-benzopyrane | |
| Geraniol | 50 |
| Geranium Essential Oil | 120 |
| Methyl dihydrojasmonate | 350 |
| Laurel Oil | 10 |
| Linalol | 150 |
| 4-(4-hydroxy-4-methylpentyl)-3-cyclohexene-1- | 90 |
| carbaldehyde | |
| 10% 1-(2,6,6-Trimethyl-1-cyclohexyl)-3-hexanol | 80 |
| 7-methoxy-3,7-dimethyl-2-octanol | 130 |
| 7-acetyl-1,1,3,4,4,6-hexamethyltetraline | 500 |
| Vanillin | 20 |
| Total | 3500 |

### Example 2

### Liquid

| | Parts by |
|---|---|
| Ingredient | weight |
| Amyl acetate | 50 |
| 3-Hexenyl acetate | 10 |
| Gamma-undecalactone | 300 |
| Ethyl butyrate | 50 |
| Allyl cyclohexylpropanoate | 50 |
| PERFUME | 150 |
| Allyl heptanoate | 300 |
| Phenoxyethyl isobutyrate | 1500 |
| 4-tert-Butyl-cyclohexyl acetate | 500 |
| Jasmolactone | 40 |
| Methyl dihydrojasmonate | 200 |
| Hexyl salicylate | 1000 |
| Veloutone | 50 |
| 2-tert-Butyl-1-cyclohexyl acetate | 2500 |
| A-Ionone | 150 |
| 2,4-Dimethyl-3-cyclohexene-1-carbaldehyde | 150 |
| Total | 7000 |

### Example 3

### Powder

| | Parts by |
|---|---|
| Ingredient | weight |
| Citronellyl acetate | 200 |
| Amylcinnamic aldehyde | 1000 |
| Hexylcinnamic aldehyde | 2000 |
| PERFUME | 100 |
| Isononyl acetate | 200 |
| Verdyl acetate | 400 |
| Verdyl propionate | 500 |
| 10% Intreleven aldehyde | 100 |
| Coumarin | 100 |
| 4-tert-butyl-alpha-methylhydrocinnamaldehyde | 1000 |
| lily aldehyde | |
| 4-tert-Butyl-cyclohexyl acetate | 1300 |
| n-heptyl cyclopentanone | 200 |
| 3-Methyl-5-phenyl-1-pentanol | 500 |
| Hexyl salicylate | 700 |
| Tetrahydromuguol | 400 |
| 10% (2E)-1-(2,6,6-trimethyl-1-cyclohexen-1-yl)-2- | 250 |
| buten-1-one | |
| (+)-(2R,4aR,8aS)-5,5,8a-trimethyldecahydro- | 150 |
| naphthalen-2-yl acetate | |
| Isomethyl-α-ionol | 300 |
| Vertofix coeur | 400 |
| TOTAL | 9800 |

### Example 4

### Liquid

| | Parts by |
|---|---|
| Ingredient | weight |
| PERFUME | 76 |
| Benzyl benzoate | 150 |
| Coumarin | 100 |
| Lavandin oil | 100 |
| Linalyl acetate | 100 |
| Patchouli oil | 65 |
| Phenylethanol | 75 |
| Linalol | 50 |
| Geranium oil Bourbon | 35 |
| Undecanal | 20 |
| Sandalwood oil | 20 |
| Anisic aldehyde | 5 |
| TOTAL | 900 |

What is claimed is:

1. A filtration device to remove a liquid from a suspension comprising:
(a) at least one concentrator filter with at least one surface and connected to a source of vacuum through an extraction tube;
(b) at least one sparging filter with at least one surface and connected to a source of compressed gas and capable of blowing gas bubbles from 10-1000μ diameter in size;
(c) fixing the filters in a configuration to allow optimal contact of surface between filters.

2. The filtration device according to claim 1, wherein the filters are a tube, cylinder, ovoid, disc, or a cuboid.

3. The filtration device according to claim 1, wherein the filters are made of polymer, metal or ceramic material.

4. The filtration device according to claim 1, wherein the filters are made of aluminum oxide.

5. The filtration device according to claim 1, wherein the suspension consists of a chemical dispersed in a liquid.

6. The filtration device according to claim 1, wherein the suspension consists of a biological entity dispersed in a nutritional media.

7. The filtration device according to claim 1, wherein the gas is air, nitrogen, oxygen, carbon dioxide or argon.

8. The filtration device according to claim 1, wherein the concentrator filter comprises a hollow perforated tube sheathed with a synthetic membrane filter.

9. The filtration device according to claim 8, wherein the sheath is made of sintered polyethylene or nylon.

10. The filtration device according to claim 8, wherein the sheath has an electrical charge that repels suspended particles.

11. The filtration device according to claim 1, wherein the filters are set apart at least 10μ and no more than 50 mm.

12. The filtration device according to claim 1, wherein a plurality of concentrator filters are assembled around a sparging filter.

13. The filtration device according to claim 1, wherein a plurality of sparging filters are assembled around a concentrator filter.

14. The filtration device according to claim 1, wherein the pore size of the filters is less than 5μ.

15. The filtration device according to claim 1, wherein the vacuum source is a peristaltic pump operating on the extraction tube attached to the concentrator filter.

16. A method of concentrating cells and organisms in a bioreactor comprising:
(a) providing a filter device according to claims 1-15;
(b) disposing the filter device in the bioreactor containing a suspension of a biological entity in nutrient media ready for concentrating;
(g) turning on gas flow to sparging filter;
(h) turning on the vacuum source attached to the concentrator filter;
(i) removing a fixed volume of nutrient media from the bioreactor.

17. A method of concentrating a chemical suspension in a reactor comprising:
(a) providing a filter device according to claims 1-15;
(b) disposing the filter device in the reactor containing the suspension of a chemical ready for concentrating;
(g) turning on gas flow to sparging filter;
(h) turning on the vacuum source attached to the concentrator filter;
(i) removing a fixed volume of liquid from the reactor.

18. A compound 1α-hydroxy-3α,6,6,9α-tetramethylperhydronaphtho[2,1-b]furan).

19. A compound 1α,11α-dihydroxy-3α,6,6,9α-tetramethylperhydronaphtho[2,1-b]furan.

20. A compound 1α,6α-dihydroxy-3α,6,6,9α-tetramethylperhydronaphtho[2,1-b]furan.

21. A compound 1α,6α,11α-trihydroxy-3α,6,6,9α-tetramethylperhydronaphtho[2,1-b]furan.

22. A method of manufacture of the compounds of claims 1-4 by fermentation of 3α,6,6,9α-tetramethylperhydronaphtho[2,1-b]furan with Fusarium lini ATCC 9593 under suitable fermentation conditions.
